(19) United States
(12) Patent Application Publication
(10) Pub. No.: US 2024/0329239 A1
(43) Pub. Date: Oct. 3, 2024

(54) STATISTICAL ACOUSTIC SENSING-BASED SYSTEM AND METHOD FOR IN-VEHICLE CHILD PRESENCE DETECTION

(71) Applicant: The University of Hong Kong, Hong Kong (HK)

(72) Inventors: Chenshu WU, Hong Kong (HK); Yi ZHANG, Beijing (CN); Weiying HOU, Hong Kong (HK)

(21) Appl. No.: 18/613,083

(22) Filed: Mar. 21, 2024

Related U.S. Application Data
(60) Provisional application No. 63/492,250, filed on Mar. 27, 2023.

(51) Int. Cl. G01S 15/04; A61B 8/08; B60Q 9/00; G01S 15/50

(52) U.S. Cl. CPC G01S 15/04; A61B 8/08; B60Q 9/00; G01S 15/50; A61B 2503/06

(57) ABSTRACT

The present invention provides a statistical acoustic sensing (SAS)-based method and system for in-vehicle Child Presence Detection. The SAS-based method comprises: transmitting acoustic signal to a subject in a vehicle cabin; receiving acoustic multipath signals scattered by the subject; and processing the acoustic multipath signals to detect presence of the subject by: extracting a plurality of channel impulse response (CIR) data from the received acoustic multipath signals; aggregating the extracted CIR data to estimate acoustic channel state information (CSI); obtaining an autocorrelation function (ACF) of the acoustic CSI based on a statistical acoustic sensing (SAS) model; and performing motion detection and breath tracking on basis of the ACF to detect presence of the subject in the vehicle cabin. The present invention can leverage in-car audio systems to detect presence of young children including newborns in an accurate and responsive manner.

### COPYRIGHT NOTICE

A portion of the disclosure of this patent document contains material, which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### CROSS-REFERENCE TO RELATED APPLICATIONS

The present application claims priority from the U.S. Provisional Patent Application No. 63/492,250 filed 27 Mar. 2023, and the disclosure of which is incorporated herein by reference in its entirety.

### FIELD OF THE INVENTION

The present invention generally relates to statistical acoustic sensing technologies. More specifically the present invention relates to a statistical acoustic sensing-based method and system for in-vehicle Child Presence Detection (CPD).

### BACKGROUND OF THE INVENTION

The ability of cars to sense, and save lives, inside a car remains to be improved. One life-critical feature that is widely missing is in-vehicle CPD. Despite remarkable advances in automobiles in recent years, unfortunately, the cases of hot car deaths are only increasing. All of these deaths could have been prevented, if the car can detect the unattended child timely and responsively alert concerned parties or take prompt actions to keep the car cool and the child safe.

Existing solutions include early systems using special sensors such as optical/weight/pressure/ultrasonic sensors, cameras, as well as recent efforts with Ultra-Wideband (UWB) or millimeter-wave (mmWave) radars, WiFi, etc. The mainstream practice mostly focuses on geometrical parameters, e.g., Time of Flight (ToF) and Doppler Frequency Shift (DFS), of a few multipath reflections around the range where a target presents. These solutions, however, suffer from different limitations. Many works focus on adult passenger monitoring, and cannot generalize well to infants and toddlers. And the sensing coverage is mostly limited to only the seats (for special seat sensors) or a certain (for cameras/UWB/mmWave radars), leading to degraded accuracy in Non-Line-Of-Sight (NLOS) scenarios and blind spots, e.g., when a child is in a rear-facing car seat, blocked by a seat, or on the car floor. More importantly, these techniques require extra hardware that is not standard offerings in today's cars to be precisely installed. This not only introduces additional hardware and manufacturing costs, but is also backward-incompatible with most of the over one billion existing cars in the world. For examples, passive infrared sensors can extend the range beyond the seats, but are limited to the Line-Of-Sight (LOS) view and sensitive to temperature changes. Camera-based systems can be accurate given good lighting conditions, but cannot see through seats, in addition to being privacy-invasive and computation hungry. Radio-based systems have been recently popular. The radar industry is promoting radar systems for CPD in new car models. UWB and mm Wave radars feature high sensing resolution, yet the coverage is limited to the Field-of-View (FoV) and the performance degrades for NLOS scenarios. Moreover, they need precise installation with wire/cable harnesses, usually on the roof of a car, to provide good coverage. With WiFi becoming prevalent for in-car connectivity, it has been exploited by the industry for CPD. Yet due to the innate limitations, it is challenging to detect vital signs of little infants using WiFi. Ultrasonic motion sensors have been used, e.g., in Hyundai cars, yet only report low detection accuracy and are being replaced. Most importantly, all these solutions will incur additional dedicated hardware and/or costly installation as being non-standard offerings in cars, preventing their wide adoption, especially for old car models. Therefore, a truly pervasive system that requires no extra hardware and works for all cars still lacks.

Acoustic signals appear to be an attractive choice for ubiquitous and practical in-car CPD. For example, an infant breathing monitoring system BreathJunior has been developed, in which nicely embeds FMCW signals into white noise for infant breathing monitoring with a limited coverage of <1 m. However, BreathJunior is not suitable for in-car CPD as it relies on a large microphone array that is unavailable in commercial cars. Therefore, there are still numerous challenges to build an accurate and robust system using a single microphone.

### SUMMARY OF THE INVENTION

One object of the present invention is to provide an accurate and robust in-car CPD system which can leverage in-car audio systems to detect tiny motions and extremely weak breathing of young children including newborns in an accurate and responsive manner.

In accordance with a first aspect of the present invention, a system for detecting presence of a subject in a vehicle cabin is provided. The system comprises: one or more transmitters, each configured to transmit an acoustic signal to the subject in the vehicle cabin; and at least one receiver configured to receive a plurality of acoustic multipath signals scattered by the subject; a controller configured to generate one or more driving signals to control the one or more transmitters to transmit the acoustic signal; and a processor coupled with the controller and configured to receive the plurality of acoustic multipath signals from the receiver and process the plurality of acoustic multipath signals to detect presence of the subject in the vehicle cabin.

In accordance with a second aspect of the present invention, a method for detecting presence of a subject in a vehicle cabin is provided. The method comprises: transmitting an acoustic signal to the subject in the vehicle cabin; receiving a plurality of acoustic multipath signals scattered by the subject; processing the plurality of acoustic multipath signals to detect presence of the subject in the vehicle cabin.

In various embodiments of the present invention, the presence of the subject is detected by: extracting a plurality of channel impulse response (CIR) data from the plurality of received acoustic multipath signals; aggregating the plurality of extracted CIR data to estimate acoustic channel state information (CSI); obtaining an autocorrelation function (ACF) of the acoustic CSI based on a statistical acoustic sensing (SAS) model; and performing one or more physiological activity monitoring on basis of the ACF to detect presence of the subject in the vehicle cabin.

In the present invention, resilience to environmental noises is achieved by orthogonal sequence for channel measurement. Sensing coverage is boosted by use of statistically leveraging all multipaths (time diversity), optimally combining multiple subcarriers (frequency diversity), and opportunistically exploiting multiple speakers (space diversity), which ultimately allows comprehensive detection in a car. Last, CPD is a time-critical mission requiring fast response (e.g., detection within 10 seconds). An instantaneous motion/breathing detector for CPD is designed based on a time-domain approach, which can detect child presence rapidly (motion in a few seconds and breathing with a minimum delay slightly exceeding one breathing cycle).

The present invention overcomes multiple challenges in translating SAS model into a practical CPD system that can reliably detect the presence of a child in a car, achieving whole-car detection with no blind spots. Importantly, it can be readily deployed in existing and emerging car models, offering the best ubiquity superior to the aforementioned other solutions.

The present invention also underpins a unified pipeline for detecting motion, estimating breathing rates, and even measuring moving speeds, truly embraces all the reflections and favors complex multipath environments, while requiring only a single microphone rather than a microphone array.

### BRIEF DESCRIPTION OF THE DRAWINGS

Embodiments of the invention are described in more details hereinafter with reference to the drawings, in which:

FIG. 1A shows a system for detecting presence of a subject in a vehicle cabin according to one embodiment of the present invention;

FIG. 1B shows an application scenario of the system in FIG. 1A;

FIG. 1C shows a flowchart of a method for detecting presence of a subject in a vehicle cabin according to one embodiment of the present invention.

FIG. 2 shows CIR measurements with and without human motion.

FIGS. 3A and 3B respectively show an ACF matrix and an extracted motion level related to cases of human motion and breathing in a bedroom.

FIG. 4A shows an experimental setup for speed estimation, FIGS. 4B and 4C show an ACF matrix and an ACF for speed estimation respectively.

FIG. 5A shows the auto-correlation and cross-correlation of a pair of exemplary Kasami sequences; FIG. 5B shows auto-correlation and cross-correlation of a pair of exemplary Kasami sequences applied with a high pass filter; FIG. 5C shows the auto-correlation and cross-correlation plotted between the original and filtered Kasami sequence; FIG. 5D shows the spectrum of traffic noise and the measured CSI after applying a high pass filter.

FIG. 6 shows the channel estimation process in the SAS-based detection system, with two speakers.

FIGS. 7A to 7C show a breathing tracking example, where FIG. 7A shows the calculated ACF matrix on different subcarriers, FIG. 7B shows the raw amplitude of good subcarriers (manually selected); and FIG. 7C shows the ACF of sensing signals (either breathing or speed) synchronized across different subcarriers.

FIG. 8 shows devices for implementing a prototype of the SAS-based detection system.

FIG. 9 shows motion detection rate and false alarm rate of the SAS-based detection system under indoor conditions.

FIG. 10 shows breathing detection error of the SAS-based detection system under indoor conditions.

FIG. 11 shows breathing detection errors of the SAS-based detection system in the three cases: there is Line-Of-Sight (LOS) between the speaker and the subject; there is no Line-Of-Sight (NLOS) between the speaker and the subject; and the subject is wearing a thick coat (w/Coat).

FIG. 12 shows an experimental setup for a feasibility study carried out with a SimNewB newborn simulator in a clinical facility.

FIG. 13 shows detected breath rates and breath rate error by the SAS-based detection system with a neonatal simulator.

FIG. 14 shows presence detection rates and false alarm rates by the SAS-based detection system for children at different ages.

FIG. 15 shows presence detection rates and false alarm rates by the SAS-based detection system for a child in different cars.

FIG. 16 shows presence detection rates by the SAS-based detection system for a child at different in-car locations.

FIG. 17A shows nine different on-seat and on-floor locations for detection coverage evaluation; FIG. 17B shows the measured detection coverage of the SAS-based detection system benchmarked with baseline.

FIG. 18 shows breath detection performance comparison between the SAS-based detection system and a state-of-the-art approach BreathJunior.

FIG. 19 shows breath detection performance of using Kasami sequence against using different CIR estimation methods.

FIGS. 20A and 20B shows motion detection and breath detection performance of SAS-based detection system evaluated using JBL Stage1 621 car speaker and Linhuipad car microphone.

FIG. 21 shows a comparable table about motion detection on various devices. an impact of background interference.

FIG. 22 shows motion detection rates of the SAS-based detection system under impact of different types of background interference.

FIG. 23 shows an impact of back ground interference level (traffic noise and human voice) on the breath rate detection error of the SAS-based detection system.

FIG. 24 shows an impact of transmitting sound level on the motion detection rate and false alarm rate of the SAS-based detection system.

FIGS. 25A and 25B motion and breath detection performance of the SAS-based detection system respectively at different frequency band.

FIG. 26 shows impact of temperature on motion and breath detection performance of the SAS-based detection system.

FIG. 27 shows an impact of the time offsets due to synchronization errors on breath detection performance of the SAS-based detection system.

### DETAILED DESCRIPTION

In the following description, a method and a system for detecting presence of a subject in a vehicle cabin and the likes are set forth as preferred examples. It will be apparent to those skilled in the art that modifications, including additions and/or substitutions may be made without departing from the scope and spirit of the invention. Specific details may be omitted so as not to obscure the invention; however, the disclosure is written to enable one skilled in the art to practice the teachings herein without undue experimentation.

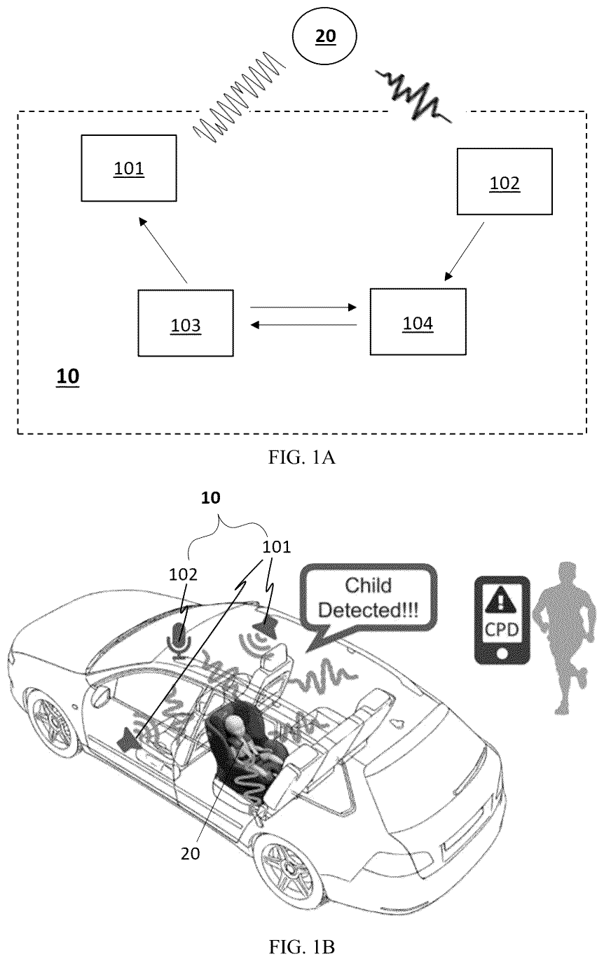

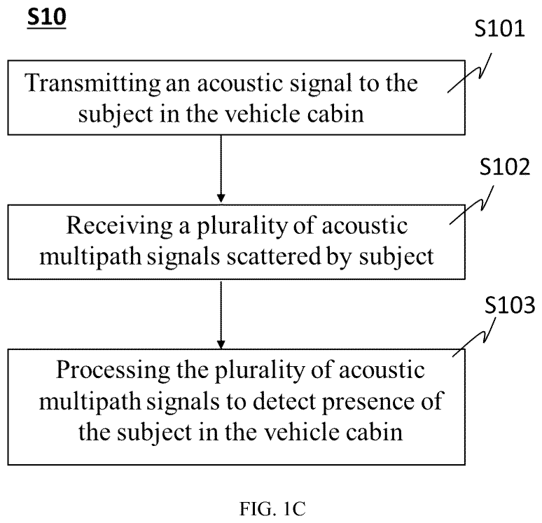

FIG. 1A shows a system 10 for detecting presence of a subject 20 in a vehicle cabin according to one embodiment of the present invention and FIG. 1B shows an application scenario of the system 10. As shown, the system 10 comprises: one or more transmitters 101, each configured to transmit an acoustic signal to the subject 20 in the vehicle cabin; at least one receiver 102 configured to receive a plurality of acoustic multipath signals scattered by the subject; a controller 103 configured to generate one or more driving signals to control the one or more transmitters; and a processor 104 coupled with the acoustic multipath channel and configured to process the plurality of acoustic multipath signals to detect presence of the subject the subject in the vehicle cabin. In some embodiments, the controller 103 and processor 104 may be integrated in an integrated circuit (IC) chip.

Accordingly, FIG. 1C shows a flowchart of a method S10 for detecting presence of a subject in a vehicle cabin according to one embodiment of the present invention. As shown, the method S10 comprises the following steps:
S101: transmitting an acoustic signal to the subject in the vehicle cabin;
S102: receiving a plurality of acoustic multipath signals scattered by subject;
S103: processing the plurality of acoustic multipath signals to detect presence of the subject in the vehicle cabin.

The presence of the subject is detected by: extracting a plurality of channel impulse response (CIR) data from the plurality of received acoustic multipath signals; aggregating the plurality of extracted CIR data to obtain an acoustic channel state information (CSI); obtaining an autocorrelation function (ACF) of the acoustic CSI based on a statistical acoustic sensing (SAS) model; and performing one or more physiological activity monitoring on basis of the ACF to detect presence of the subject in the vehicle cabin.

CSI, also known as Channel Frequency Response (CFR), is the frequency-domain counterpart of CIR. CSI for an acoustic multipath channel of frequency fat time t is denoted as the following equation:

$$H ( f , t ) = {\sum }_{r = 1}^{R} {a}_{r} ( t ) exp ( - j 2 π f {τ}_{r} ( t ) ) \qquad ( 1 )$$

where aᵣ (t) and τᵣ (t) are the complex amplitude and propagation delay of the r-th reflection path, respectively, while R denotes the total number of paths.

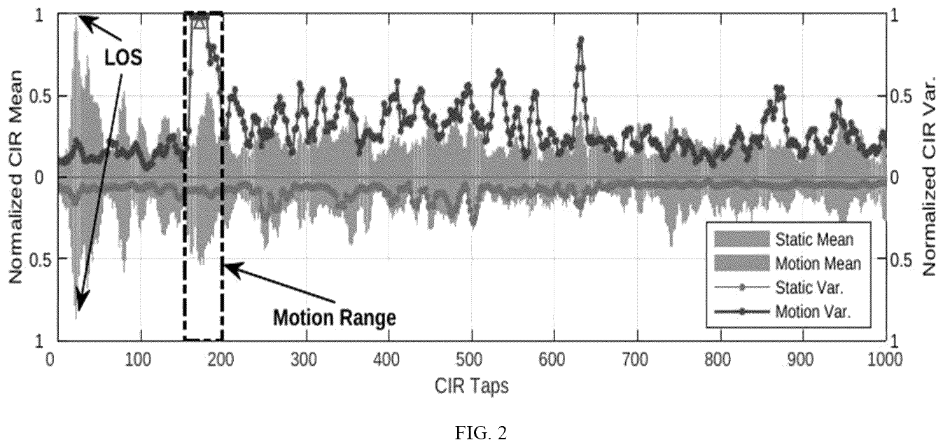

While previous works mostly only focus on reflections from the range of interest and segment others out, the present invention properly aggregates multipath distortions caused by a target at a certain range and extract useful information for sensing. As shown in FIG. 2, while a human target contributes the strongest reflection at the CIR taps corresponding to her/his range (i.e., 1.25 m), the CIR taps up to 7 m after that range are also altered remarkably. In comparison, the CIR taps are mostly noises in the empty case without human presence. That means a target at a certain range not only alters the reflection around that range, but also distorts multipath signals arriving later to a considerable extent.

From a rich-scattering perspective, each reflection path can be treated as a scatterer that scatters the incoming energy back to the receiver (i.e., microphone). Thus, it can infer the following equation:

$$H ( f , t ) = {\sum }_{i ∈ {R}_{D}} {H}_{i} ( f , t ) + {\sum }_{j ∈ {R}_{S}} {H}_{j} ( f , t ) + N ( f , t ) , \qquad ( 2 )$$

where Hᵢ(f,t) denotes the component contributed by the i th scatterer, N(f,t) is the noise term with variance σ_{N}², and R_{S} and R_{D} denote the set of static and dynamic scatterers, respectively.

Assuming all scatterers are statistically independent of each other, each with the same variance σᵢ²(f) and approximately zero means, the ACF of H(f,t) obeys the 0^{th}-order Bessel function of the first kind. That is, denoting ρᵢ (f,τ) as the ACF of Hᵢ (f,t) with time lag τ, it can be inferred that ρᵢ (f,τ)=J₀(kviτ), where

$${J}_{0} ( x ) = \frac{1}{2 π} {\int }_{0}^{2 π} exp ( - jx cos ( θ ) ) d θ ,$$

vᵢ is the moving speed of Hᵢ(f, t), k is the wavenumber.

Suppose there is one single moving target, and thus all dynamic scatterers have approximately the same speed v, vᵢ≈v, ∀i∈R_{D}. This assumption is realistic because, for human subjects, the torso scatterers dominate others and have a similar speed. Then motion detection may be performed by: calculating a channel gain of the acoustic CSI from the ACF; comparing the channel gain against a threshold; determining that motion is detected if the channel gain is equal or greater than the threshold.

More specifically, the ACF ρ(f, τ) of H(f, t) can be associated with the target's moving speed v as follows:

$$For τ \neq 0 , \\ \begin{matrix}ρ ( f , τ ) = \frac{{\sum }_{i ∈ {R}_{D}} 2 π {σ}_{i}^{2} ( f ) + {σ}_{N}^{2} ( f ) δ ( τ )}{{\sum }_{i ∈ {R}_{D}} 2 π {σ}_{i}^{2} ( f ) + {σ}_{N}^{2} ( f )} {J}_{0} ( kv τ ) \\ {=}^{△} g ( f ) {J}_{0} ( kv τ )\end{matrix} , \qquad ( 3 )$$

where δ( ) is the Dirac's delta function and g(f) is defined as the channel gain of H(f, t).

The channel gain is then associated with the ACF by: g(f)=\tilde{ρ}(f, t)=ρ(f, τ)+n(f, τ), where \tilde{ρ}(f, τ) is the sampled ACF calculated from a time series of CSI measurements with the noise term n(f, τ).

From Eq. (3), it can be inferred that g(f)=lim_{τ→0}ρ(f, τ) since lim_{τ→0}J₀(kvτ)=1. Hence, given a sufficient CSI sampling rate Fₛ, g(f) can approximate as the value of the first tap of the ACF, i.e.,

$$g ( f ) = \tilde{ρ} ( f , τ = 1 / {F}_{s} ) , \qquad ( 4 )$$

If any motion presents, the value of g(f) is greater than zero; otherwise g(f)→0. In other words, the defined channel gain g(f) is used as a sensitive and robust indicator for acoustic motion detection.

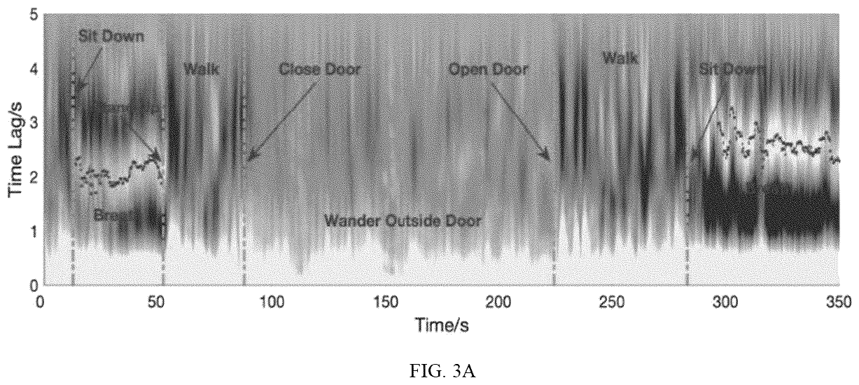

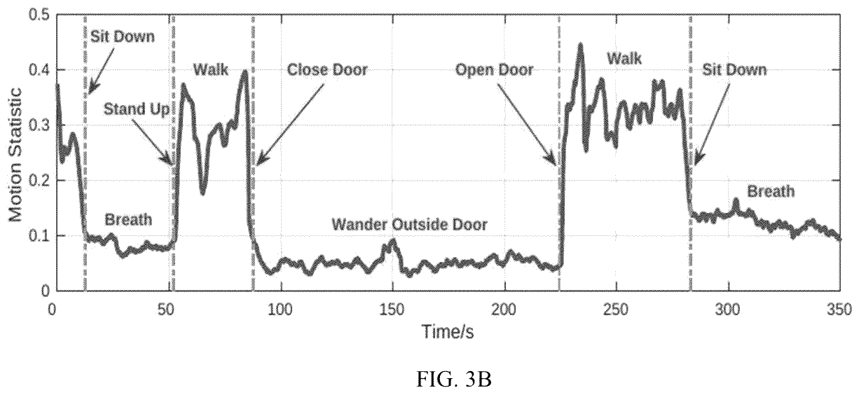

FIGS. 3A and 3B shows an example of g(f) in the case of human presence and absence, respectively. As seen, there is a clear gap between the empty level and the values for motion. Additionally, g(f) exhibits larger values when the motion is stronger/closer, implying that it also indicates motion strengths.

Breathing signals, which are periodic signals induced by repeated chest movements, are detected from the ACF. If a breathing signal is captured by CSI, the ACF will observe a prominent peak at the time lag Tb corresponding to the cycle time, as shown in FIG. 3A. Then breathing tracking may be performed by: searching peaks in the autocorrelation function (ACF) over time corresponding to a cycle time of breathing; and determining that breathing is detected and tracked if the peaks are found. That is, by finding time lags of these peaks over time, breathing rates of the subject (e.g. 60/τ_{b} BPM (breath per minute)) can be tracked. Note that as a time-domain approach, ACF in principle is faster for breathing estimation compared to spectrum-based approaches, which usually require a much longer window to yield better frequency resolution.

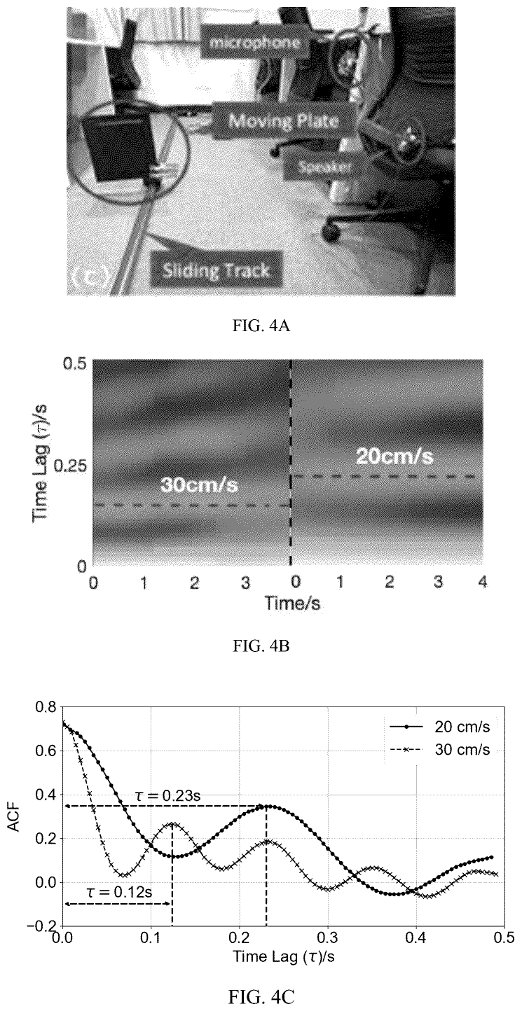

As indicated by Eq. (3), the ACF of CSI is a function of speed v, which underpins a statistical approach entirely different from the Doppler effect for speed estimation. Specifically, as shown in FIG. 4B, the shape of the ACF ρ(f, τ) resembles the Bessel function J₀(x) with x=kvτ, meaning that it can estimate the speed by aligning ρ(f, τ) with J₀(x). Assuming x₀ is the constant value corresponding to the first peak of J₀(x), then the moving speed v can be calculated as

$$\hat{v} = \frac{{x}_{0}}{k {τ}_{s}} = \frac{{x}_{0} λ ( f )}{2 {πτ}_{s}} ,$$

where τₛ is the time lag corresponding to the first local peak of the ACF ρ(f, τ) and λ(f) is the wavelength of subcarrier f. FIG. 4A illustrate a setup for speed estimation demonstration. As shown, a speaker and a microphone are used to estimate speed of a plate moving along a track programmed with different speeds. It can be seen from FIGS. 4B and 4C that the ACF of the CSI measurements reacts to the moving speed faithfully as the above equation implies.

As seen, a peak in the ACF can either indicate a speed signal or a periodic signal. However, the peak locations for breathing (e.g., 1-5 s for breathing rates 60-12 BPM) are usually of magnitude longer than those for speed (e.g., <0.5 s for 0.5 m/s using 10 kHz sound, and the faster the speed, the smaller the delay), a sufficient difference to determine whether to estimate breathing or speed.

Several unique characteristics of sound waves make CSI measurements (or channel estimation) particularly challenging. First, the sound wave speed is orders of magnitude slower than that of light and EM waves, which imposes limitations on the max possible CSI sampling rate of the acoustic channel. For example, given the in-air sound speed of around 343 m/s, the propagation delay of a path of 7 meters in length will be greater than 20 ms, requiring a minimum channel measurement internal larger than 20 ms to avoid signal mixture. Second, acoustic sensing is vulnerable to environmental sound interference, especially when it is limited to a frequency band under 24 kHz on commodity devices. Ambient interference like the human voice, music, and natural sounds, can smear channel measurements for certain frequency bands. Moreover, concurrent sensing signals transmitted on multiple speakers, if used, may also interfere with each other.

To address above challenges, pulse coded modulation (PCM) samples are generated from a pseudo-noise sequence and applied on the speaker for channel estimation (or CSI estimation. In other words, the acoustic signal is PCM signal modulated with the pseudo-noise sequence. The PN sequence may be selected from, but not limited to, m-sequence, Golay sequence, GSM training sequence, and ZadoffChu (ZC).

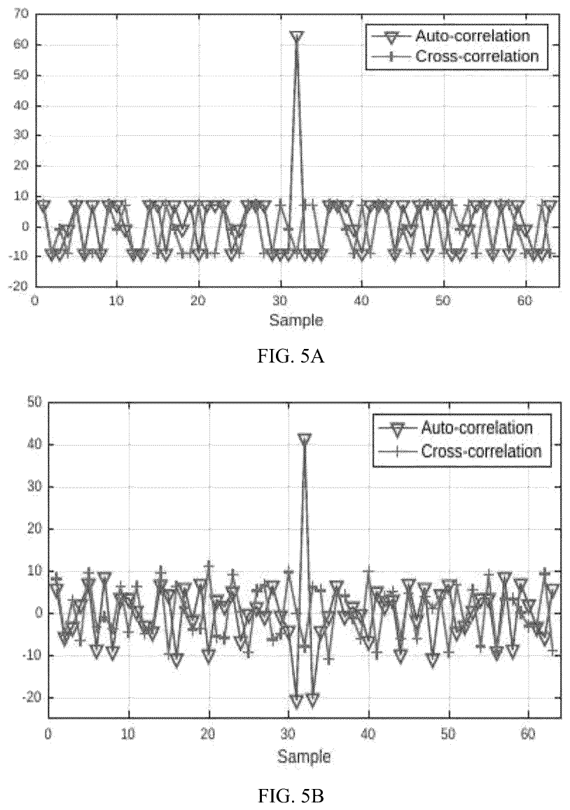

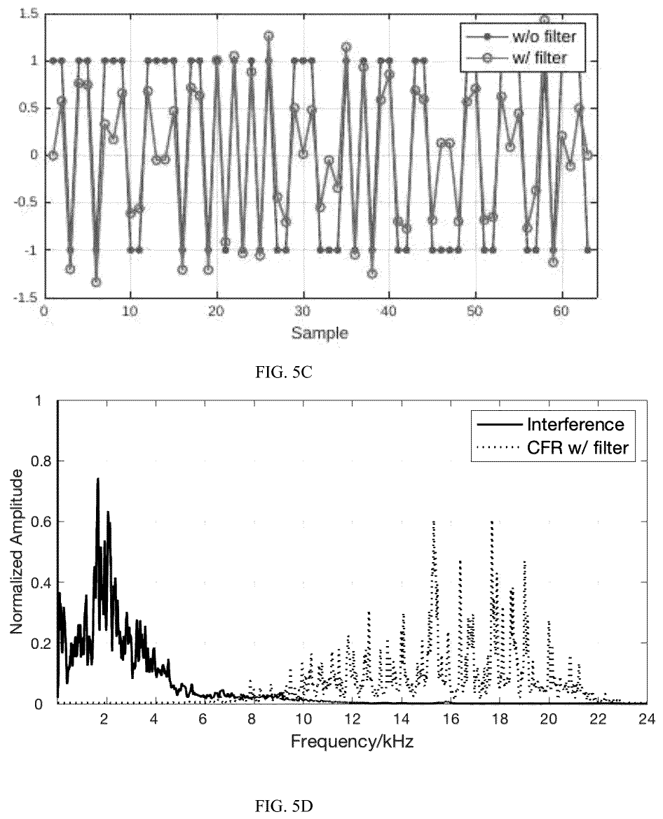

Preferably, Kasami sequence is selected because of its superior properties of orthogonality and noise tolerance. FIG. 5A shows the auto-correlation and cross-correlation of a pair of exemplary Kasami sequences with period 2⁶−1. The auto-correlation produces an impulse-like signal with minor side lobes, while the cross-correlation only produces minor values that are much smaller than the impulse of auto-correlation. Note that the present disclosure is not limited to a particular channel estimation technique, but can work with any approach, including the widely used FMCW, that provides effective CSI.

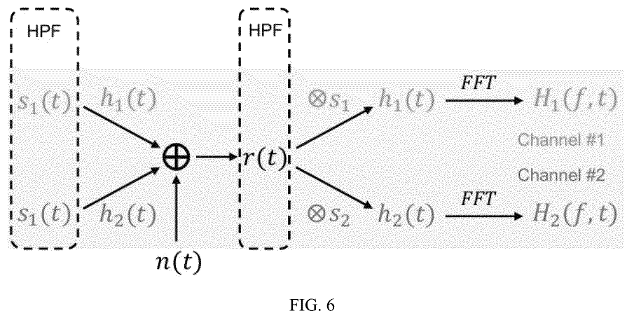

FIG. 6 shows the channel estimation process in the SAS-based detection system, with two speakers. Two orthogonal Kasami sequences s₁ and s₂ are generated with the same length and periodically transmit them on both speakers simultaneously. The transmitted sequences undergo different time delays and attenuation before being captured by the microphone. On the receiver side, the microphone recordings are correlated with s₁ and s₂ separately to get CIR streams of the two channels, and slice them into segments with the same length as s₁ and s₂, resulting in the CIR estimates h₁(t) and h₂(t). Then, h₁(t) and h₂(t) are converted into the frequency domain by performing Fourier transform and obtaining the CSI H₁(f, t) and H₂(f, t).

Since a correlation operation is equivalent to a conjugate multiplication in the frequency domain, the measured CSI \tilde{H}(f) using Kasami sequence can be represented as

$$\begin{matrix}\tilde{H} ( f ) = [ S ( f ) \cdot H ( f ) + N ( f ) ] \cdot {S}^{*} ( f ) \\ = {‖ S ( f ) ‖}_{2} \cdot H ( f ) + N ( f ) \cdot {S}^{*} ( f )\end{matrix} , \qquad ( 5 )$$

where H(f) denotes the ideal CSI, and S(f) and N(f) are the frequency-domain representations of Kasami sequence and sound noises respectively. S(f) is a wideband signal spanning over the whole spectrum, and S*(f) is its conjugate. The term ∥S(f)∥₂·H(f) approximates to a scaled version of H(f). An example of the measured CIR is shown in FIG. 2.

There are two issues with the above channel estimation process. First, the Kasami sequences composed of 1's and −1's with sharp transitions in between can be intrusive to human cars. Second, by transforming CIR, the obtained CSI spans the full spectrum, which might be polluted by the ambient sound noises, especially on the audible frequency band.

To circumvent these problems, high-pass filtering is applied on both the transmitted and received signals. The passband can be set flexibly, and the SAS-based detection system can work reliably even with only the inaudible pseudo-ultrasound band, e.g., above 18 kHz. Here, an empirical passband of 10 kHz is used as an example.

When the filter is applied on the transmitter side, as shown in FIG. 5B, the binary values on the time domain signal are softened, and the output sound contains fewer intrusive bursts. The major concern is whether this filter operation breaks the auto-correlation and cross-correlation properties of Kasami sequences or not. To validate, the auto-correlation and cross-correlation are plotted between the original and filtered Kasami sequence in FIG. 5C. It can be seen that, after applying the filter, the auto-correlation still observes an impulse (with a decrease in SNR) while the cross-correlation approximates the noise.

On the receiver side, the term N(f). S*(f) in Eq. 5 is eliminated by high-pass filtering. This is because typical daily sound interference, such as traffic and human voice, mostly occurs in the frequency band below 10 kHz.

FIG. 5D shows the spectrum of traffic noise and the measured CSI after applying the HPF. As seen, the noise is successfully removed. Meanwhile, fewer subcarriers are left for sensing because of the filtering, motivating us to maximize the sensing signals.

Due to hardware imperfections and software latency, however, the speakers and microphones are not perfectly synchronized, which makes it difficult to measure accurate channel response. Fortunately, synchronization errors only introduce phase offsets in CSI, which does not affect the detection accuracy because CSI is measured consecutively without blanks in between and the amplitude is only used.

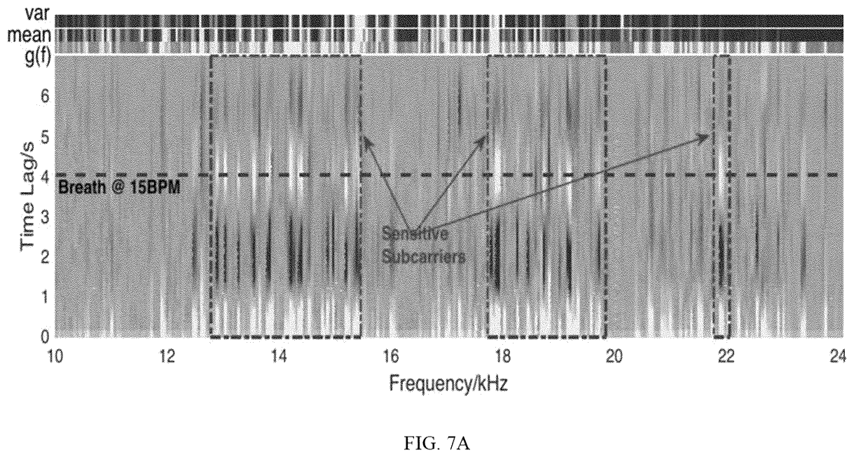

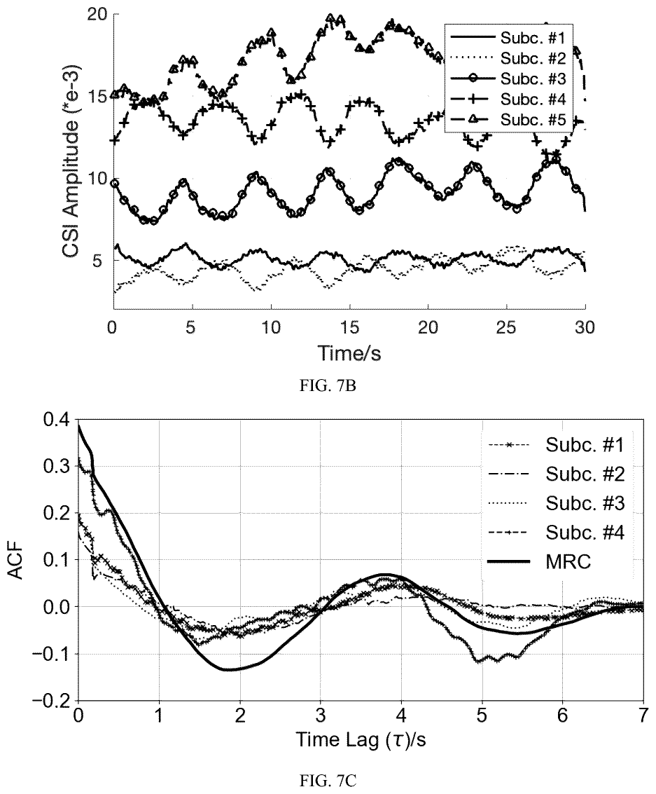

Sound reflection off human bodies is considerably weak, a major reason confining the coverage of human-centric acoustic sensing. The problem is aggravated when the target subject is an infant/toddler in CPD applications. FIGS. 7A to 7C demonstrate a breathing example, with the calculated ACF matrix on different subcarriers in FIG. 7A and the raw amplitude of good subcarriers (manually selected) in FIG. 7B. Some subcarriers capture dominant breathing signals, while others merely observe noises, even in such an example with strong breathing signals. It is also noticed that, because of the complex multipath propagation, the most sensitive subcarriers can vary over time randomly. Therefore, it is critical to dynamically find the best subsets of subcarriers and effectively combine them to maximize the signal SNR.

In some embodiments, subcarrier diversity, a phenomenon attributed to frequency selective fading, is exploited to optimize the receiving SNR for better coverage. More specifically, a maximal ratio combining (MRC) approach is employed to combine multiple subcarriers optimally to utilize all multipath signals effectively. Since the noise terms on different subcarriers are statistically independent, the signal SNR by MRC can be maximized as

$$\\ \begin{matrix}\hat{ρ} ( τ ) = {\sum }_{f ∈ F} w ( f ) \tilde{ρ} ( f , τ ) , & ( 6 )\end{matrix}$$

where \hat{ρ}(t) is the combined ACF, w(f) denotes the normalized weight for combining subcarrier f (i.e., Σ_{f∈F}w(f)=1), and F is the set of all subcarriers. The optimal weight w(f) should be linearly proportional to the gain on each subcarrier.

Following, the normalized g(f) is adapted, defined in Eq. (4), as the weight w(f) in the SAS-based detection system. Note that, some intuitive criteria commonly used like mean/variance of CSI amplitude cannot serve as the optimal weights for MRC, as they are not linearly proportional to the gain g(f) and subcarriers with higher amplitude means/variances do not necessarily better capture the sensing signals, as shown by FIG. 7A.

MRC can be applied here because, by taking the ACF, the sensing signals (either breathing or speed) are synchronized across different subcarriers (FIG. 7C). It cannot be done directly on the raw amplitude due to the considerable phase offsets of breathing/speed signals on different subcarriers, as demonstrated by FIG. 7B. In case multiple speakers are available, the subcarriers on different speakers can be combined similarly, and again, asynchronization among different speakers is not an issue. By combining them, the SNR can be boosted and extend the sensing coverage.

In some embodiments, the subject is a child or an infant, and the one or more physiological activity monitoring may include motion detection and breath tracking such that the SAS-based detection system can be used as a CPD system.

For motion detection, the gains g(f) is averaged across all subcarriers and obtain

$$\\ \overline{g} = \frac{1}{\vert F \vert} {\sum }_{f ∈ F} {}_{} g ( f ) .$$

Then given a preset motion threshold ε, the system detects motion at any given time t if g̅(t)>ε; otherwise no motion presents. Equally averaged g̅ is used instead of using MRC because the averaged values across all subcarriers with equal gains will approximate zero in absence of motion, allowing us to find a generic threshold ε for different environments and cars.

For tracking breathing, there is need to find whether there exists a dominant peak in the enhanced sensing signal \hat{ρ}(t). To achieve so, similar criteria is adopted in for peak finding. Basically, the peak prominence is examined, width, and amplitude to identify potential peaks. Then, the peak location is checked to sift out those beyond the typical range of human breathing rates, e.g., 10-60 BPM. The motion level g̅ against the peak value is compared as there will be unlikely breathing if the motion level is way larger than the peak value. Once the peaks corresponding to breathing are found, the peak location τ_{b} will be estimated and accordingly derive the breathing rate.

In real-time CPD, a sliding window is employed on the continuous CSI to calculate the ACF. A shorter window of CSI (e.g., 1 s) is employed for calculating the ACF for motion detection to make it more responsive while saving computation. While for breathing, a minimum window larger than a typical breathing cycle (e.g., 6 s, which can be shorter for children who usually have higher breathing rates) is desired. As motion is more common and the computation is more efficient, only further perform breathing estimation is performed when no motion can be detected. Note that the system can output detection decisions as fast as every CSI sample, or at a predefined lower rate, e.g., every 1 second, to save energy. Once the time series of motion/breathing decisions are obtained, and they are checked within a certain window, e.g., 5 s, and child presence is claimed if there is a certain amount of motion/breathing detection, e.g., >30% of the window.

### Evaluation

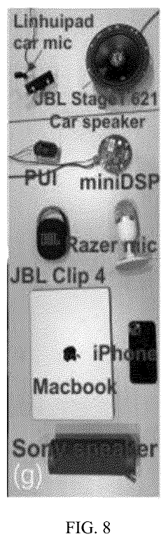

For performance evaluation, a prototype of the SAS-based detection system is implemented using a programming audio prototype, which consists of a MiniDSP UMA-8SP USB microphone array with 7 built-in Knowles SPH1668LM4H microphones (only one of them is used) and PUI Audio AS07104PO-R speakers connected to the MiniDSP board via cables, as shown in FIG. 8. The commodity devices used in performance evaluation includes JBL Stage1 621 car speaker and Linhuipad car microphone, JBL Clip 4 speaker, Sony SRS-XB23 speaker, Razer Seiren Mini Mic, and speakers and microphones on Macbook and iPhone, etc. Again, only one single microphone is used throughout experiments, even if more are available. This prototype is connected to either a computer or Raspberry PI 3 Model B+. Software: Signal generation and transmission as well as all algorithms is implemented with MATLAB mainly for benchmark analysis. An end-to-end prototype of the system is built to run in real-time using Python 3.9, which can run on embedded devices (Raspberry PI in our case).

A longer period of Kasami Sequence allows higher SNR for channel estimation, which, however, creates an immediate conflict with sampling rates. To tradeoff, a sequence of period 2¹⁰−1 modulated is used into 0.02 s, which allows a desired sampling rate of 50 Hz to use in the SAS-based detection system. By default, 3 seconds of CSI is used for motion calculation and use 8 seconds for breathing rate estimation.

By applying a high-pass filter, most of the daily environmental noises are eliminated. However, if there are sharp and short impulse-like noises (e.g., horn honk/beep), the impacts may go above 10 KHz and cause false motion detection. That these kinds of sharp noises will impose a sudden change in the CSI amplitudes, which translates into a special ACF pattern, which linearly decreases first and then linearly increases (See Appendix A.3 for more details). Therefore, a detector is designed to identify this linear decrease-then-increase pattern and skip CPD during the interfered period. By doing so, the SAS-based detection system becomes immune to sharp noise like horn beep, an important feature making it more practical. Although this would reduce the effective protection time (the system is not working in presence of such noises), the impact is minimal because these noises are usually short (˜1 s) while the SAS-based detection system detects so rapidly that it can find a period for detection.

The prototype is first evaluated with comprehensive indoor experiments to validate motion detection and breathing estimation. Motion and presence ground truths are manually labelled. Ground truth for adult breathing is measured by Plux piezoelectric Respiration (PZT) sensor. Infant simulators (e.g. SimNewB) have a preset fixed breathing rate. The ground truth breathing rate of children participants did not record as it is difficult to have their cooperation.

Detection rate (DR) and false alarm rate (FAR) are used as the evaluation metrics for motion, breathing, and overall presence detection, while the mean absolute breathing rate error is evaluated. To show the system's performance under extreme responsiveness constraints, by default a 2-second window is used for the decision. A higher DR is expected if a longer decision window is applied. Also, only one speaker is used for evaluation unless otherwise specified. Using more speakers is expected to provide larger coverage.

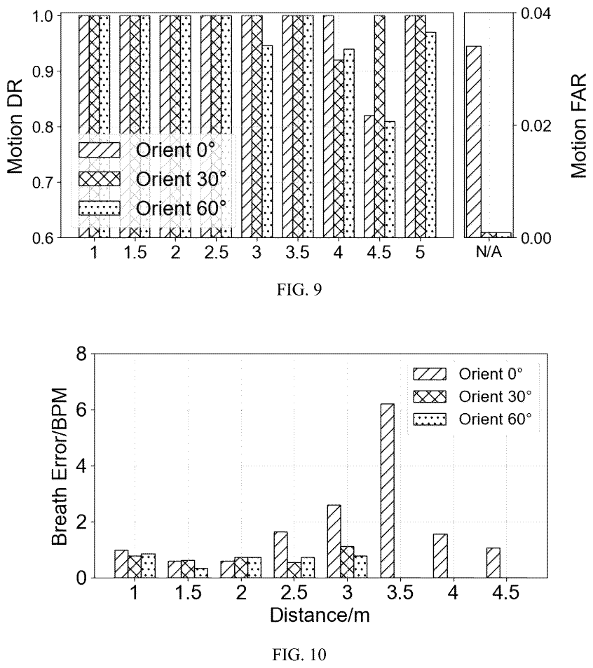

The motion detection performance is evaluated in a 7 m×5 m conference room. One microphone and one speaker are set up in the corner. The room is in an office building, with constant noise from the central fan and occasional footstep sounds when people pass by the outside corridor. An adult is asked to sit in a chair, at various distances from 1 m to 5 m, and only move his one hand slowly to mimic the tiny motion of a child. The system is also tested with the speaker facing different angles with respect to the subject. As shown in FIG. 9, under indoor conditions, the SAS-based detection system achieves an average DR of 98.1%, which maintains 94.1% even when the user is 4˜5 m away from the speaker and microphone, while the FAR is only 1.1%. The performance degrades slightly when the subject is at a distance and at an angle of 60°. Note that the motion DR is almost 100% when the user is within 3.5 m, a sufficient distance to cover a typical car.

The breathing estimation performance is evaluated in the same environment. First, the system is also tested with an adult subject at different distances, sitting still in a chair.

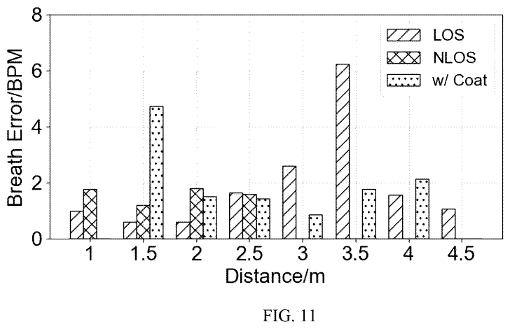

As shown in FIG. 10, a mean absolute error of 0.88 BPM within the distance of 3 m is achieved, including all orientations. More importantly, the SAS-based detection system can detect breathing rate at a range as far as 4.5 m, with a slight increase in breathing rate error. The case is also evaluated when there is no Line-Of-Sight (LOS) between the speaker and the subject, as well as the case when the user wears a thick down jacket. As portrayed in FIG. 11, even when a user is wearing a thick coat, the SAS-based detection system can still pick up the breathing rate at distance up to 4 m. When the speaker is blocked, the maximum range of breathing estimation decreases to 2.5 m, still more than enough to cover an entire car. Note that the accuracy under occlusion is not necessarily lower than that for LOS cases since the SAS-based detection system embraces all multipath reflections to significantly enhance the NLOS scenarios, which may experience richer multipath effects.

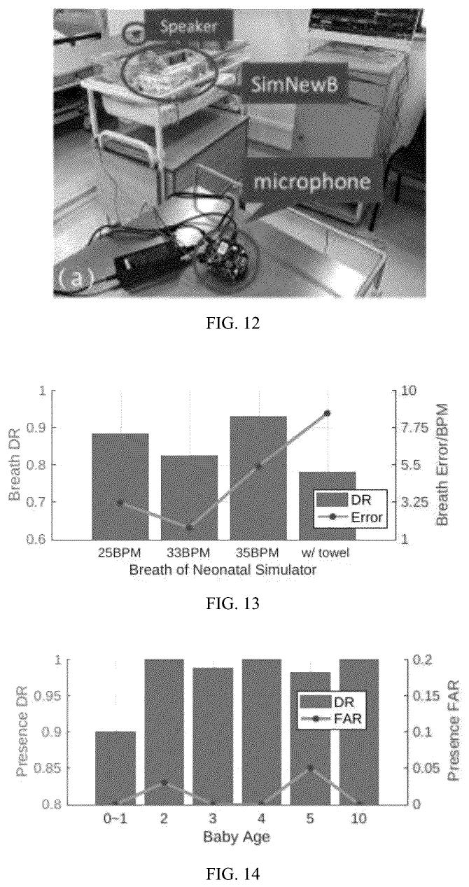

A feasibility study is carried out with the SimNewB newborn simulator in a clinical facility, which features tens of beds and has continuous machinery and HVAC noises. The experimental setup is illustrated in FIG. 12. During the tests, the laboratory technician randomly set the breathing rate of the newborn simulator. As shown in FIG. 13, the SAS-based detection system can detect the newborn simulator's very weak breathing reliably, achieving an average detection rate of 87.8% with a mean error of 3.43 BPM, which decreases to 78.0% with an increased mean error of 8.6 BPM when the newborn is covered with a blanket. Note that exhaust various breathing rates did not exhaust due to limited access to the facility and SimNewB during the pandemic time, yet it is believed the results already show the capability to detect a newborn's breathing at a distance. The impact of breathing rates later will be studied. In another test case, the neonatal simulator is configured to move her forearms, for detecting the motion for 100%.

A real-world CPD study is conducted with young children in different cars and parking scenarios, such as parking lots, roadside parking, garage, etc. 15 young children are recruited and perform CPD in 7 different cars including sedan and SUV.

For each child, cases of different locations are tested, with either forward-facing or rear-facing car seats as regulated. For older children who can sit/crawl independently, cases of seats without the baby car seat are also tested. All the children wear their regular winter coats. Motion (awake) cases are tested for every child and evaluate breathing for children who are able to get asleep (or stay very quiet) during the test. The data collection for each child lasts about 30-60 minutes. During tests, the car is parked and locked with windows closed, the typical scenario that hot-car deaths may occur. There are cars parking around and/or passing by, and parents and the experimenters talking/standing/walking around the car. There are frequent traffic noises during most of the tests, done in central downtown Hong Kong. In total, 15 children (aged 7 months, 12 months, 18 months, 2 (2×),3 (4×),4 (2×),5 (3×), and 10 years old, respectively) tested in 7 different cars, including Lexus LS430, BMW 330, Mercedes-Benz C200, Mercedes-Benz S320, Tesla Model 3, Honda Jazz, Nissan Serena. One or two speakers is used for the real-world study, considering not all cars have four or more, and always use one single microphone. In most cases, the LOS condition is occluded, provided that the devices are installed in the front row while the kids are seated in the back.

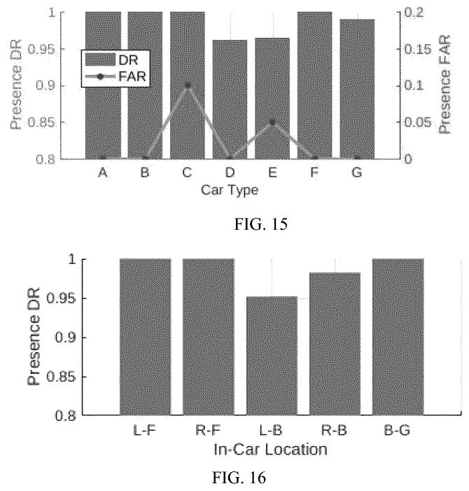

The overall presence DR is mainly focused for this CPD test. FIG. 14 shows that the SAS-based detection system achieves an average DR of 98.8% with an average FAR of 2.1% for all age groups of children. As expected, the DR for infants (one 7-month-old and one 12-month old in our experiments) is relatively lower than older kids, but is still around 90%. The high performance is consistently achieved across different cars, varying from 95% to 100% with marginal differences, as portrayed in FIG. 15. The FARs in FIG. 14 vary slightly because different kids are tested in different cars that have different enclosure materials and in car noise levels. False negatives are most likely to occur when there is a lack of awake motion and the infant's breathing is extremely weak.

Furthermore, the performance is analyzed at different in-car locations. As shown in FIG. 16, the results are grouped based on where the child seats, i.e., driver seat (L-F), passenger seat (LR), two back seats (L-B and R-B), as well as the case when the child is on the back row floor (B-G). As seen, the SAS-based detection system maintains a consistently high DR and low FAR across different locations. Overall, the results demonstrate the SAS-based detection system's remarkable performance in real-world scenarios, promising its potential for practical adoption.

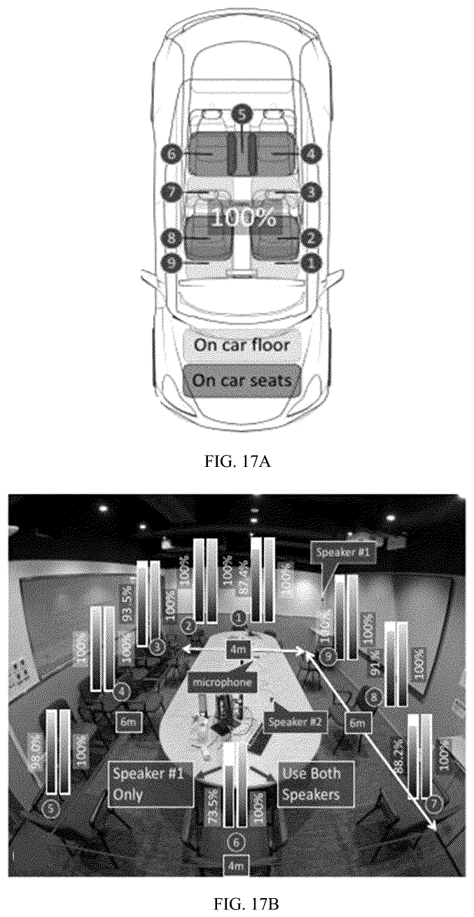

To further understand the detection coverage in a car, a small toy car is used to simulate tiny motions at nine different on-seat and on-floor locations as shown in FIG. 17A. Two speakers are installed on the left and right front doors, respectively. As depicted in FIG. 17B, the SAS-based detection system achieves a 100% DR for all the 9 testing locations, using either two speakers or only one single speaker on the left or the right.

It is also studied whether increasing number of speakers can increase the sensing coverage of the SAS-based detection system. A case study is presented with two speakers in a meeting room, where an adult sits in a chair moving one hand. As portrayed in FIG. 17B, while the coverage with a single speaker is already good, by adding one speaker, a 100% motion DR is achieved through the 6 m×4 m area. The system didn't continue with more speakers as the system already covers the entire room using two.

Besides the high accuracy, it is also critical to study false alarms, especially over a long period in diverse noisy environments like busy streets, noisy garages, etc. It is noted that the above real-world experiments were conducted in noisy urban areas (including noisy garages, busy streets, parking lots next to highways, etc) in downtown Hong Kong in the presence of cars, sirens, pedestrians, etc. To further understand the performance in different environments, it is carried out a relatively long-term evaluation in the busy Beijing City. The car is parked, without kids inside, in a busy garage and a crowded street for about 10 hours, respectively. A false alarm is reported if motion is detected for over 10% of the time for a sliding window of 2 s. The results show that the SAS-based detection system observes a FAR of 0.12% in the garage and 0.28% for the roadside parking case. In practice, a CPD system may not need to run for a long time, but perhaps only for a few minutes after the car is parked and locked, which will further reduce the chance of observing false alarms.

As a time-critical task, the detection latency of the SAS-based detection system is analyzed. To do so, the delay of the first decision for each test is evaluated. A 3 s window for ACF calculation is used for motion and an 8 s window for breathing, and then use another 2 s window for presence detection. Hence, the minimum delay will be 5 s if motion is detected and 10 s if there is no motion but breathing. With this configuration, the results show that the SAS-based detection system can output the first detection within 5.7 s for 81.9% of the time, 11.2 s for 95.2%, and 15.2 s for 98.8%. The minimum delays and thus the overall latency can be reduced by using a shorter window (e.g., 1 s) for motion detection, the most common case for CPD.

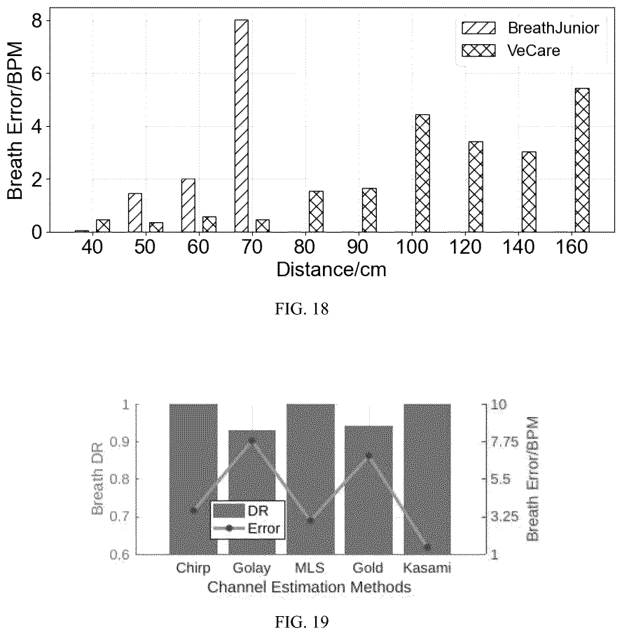

The SAS-based detection system is compared with the state-of-the-art approach BreathJunior, the closest to our work which successfully uses white noise for infant breathing monitoring. BreathJunior is implemented and performed comparison experiments using an infant simulator. The results demonstrate that the SAS-based detection system outperforms BreathJunior in both accuracy and coverage. As shown in FIG. 18, the maximum distance BreathJunior achieved is 70 cm (with a considerable error of 8 BPM), while the SAS-based detection system goes to 1.6 meters under the same condition, which is 2.3× improvement. In addition, while BreathJunior is accurate within 0.5 m, the breathing estimation error quickly increases at a distance of 70 cm. In comparison, the SAS-based detection system maintains a breathing rate error below 2 BPM at a distance of 90 cm, smaller than the error BreathJunior experiences at 60 cm.

As said, the SAS-based detection system can work with any channel estimation methods that output CIR. The performance of using Kasami Sequence against using different CIR estimation methods are compared, including chirp signals (FMCW), Golay Sequence, MLS, Gold Sequence. As shown in FIG. 19, while all these methods produce a high DR above 90%, Kasami Sequence demonstrates its superior performance with the lowest breathing rate error and the highest DR.

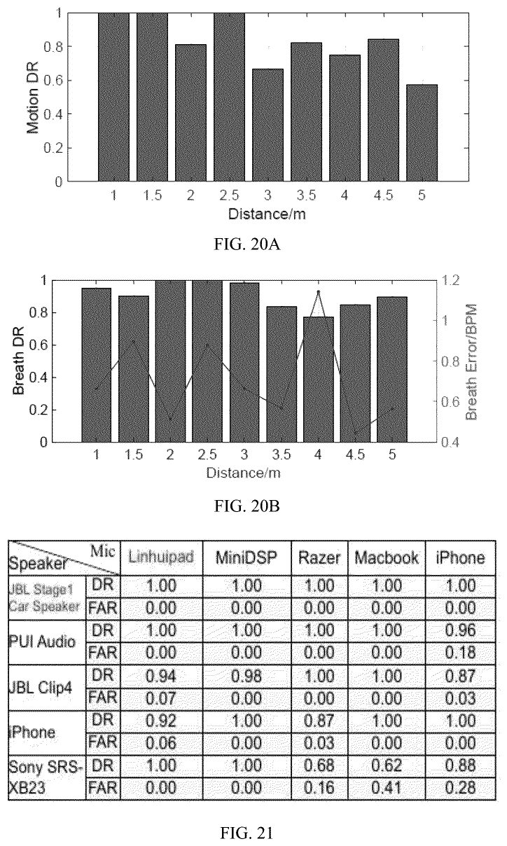

The SAS-based detection system's performance is examined on different devices. The SAS-based detection system is evaluated using JBL Stage1 621 car speaker and Linhuipad car microphone, both adopted in existing automobile audio systems. As shown in FIGS. 20A and 20B, the SAS-based detection system maintains high performance and large coverage. The system is further tested with motion detection at 2 m on various speaker/microphone combinations as summarized in FIG. 21, which indeed show device diversity yet good performance retains in most cases.

The impacts of various factors and validate the robustness of the SAS-based detection system are evaluated. For more controllable data collection, the infant simulator is used instead of real babies for this study, and focuses more on breathing estimation.

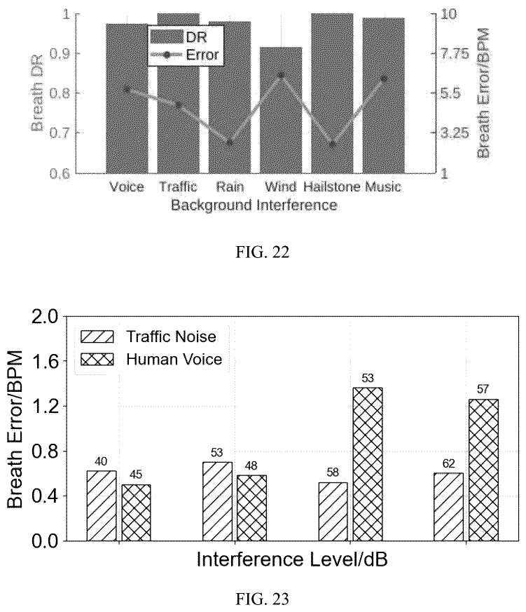

The impact of background sound interference of different types is studied, including human voices, traffic noise, rain sound, wind sound, hailstone sound, and music. To better control the experiments, sound files of these noises are downloaded and play them through a loudspeaker around 50 dB next to the SAS-based detection system. As shown in FIG. 22, the SAS-based detection system maintains high accuracy regardless of different types of natural sound interference, with only marginal differences among them. This has also been partly verified in real-world testing where the system is tested under real environments with all different ambient noises are tested.

The performance is also evaluated under various background noise levels. Traffic noise and human voices are also focused for this test. Sound files of noise is played through a loudspeaker at various powers and distances and record the actual sound level received at the microphone. As shown in FIG. 23, the BPM error increases with higher surrounding noises, especially over 50 dB level.

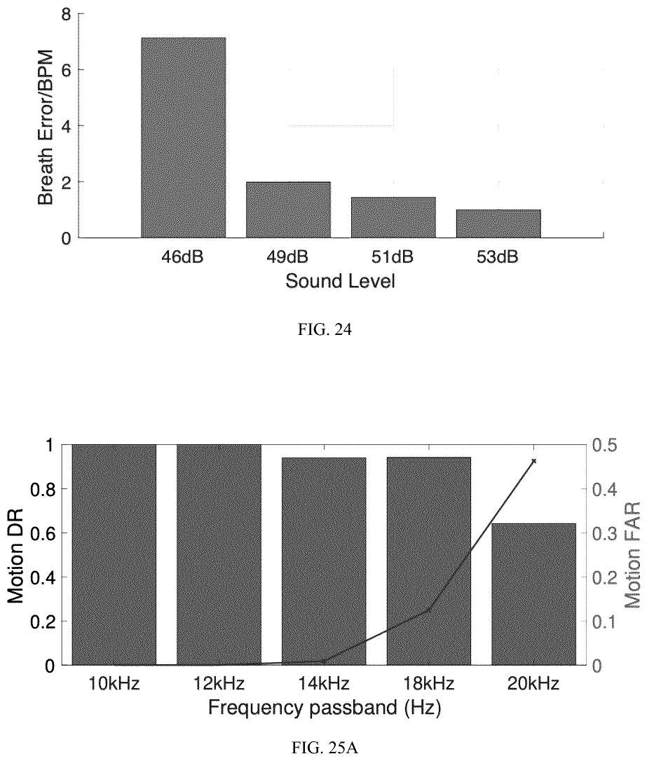

The transmitting power of the speaker can affect performance. To verify this, the transmitted sound is varied from 46 dB to 53 dB and evaluate the breathing estimation error accordingly. As seen in FIG. 24, the breathing rate error quickly drops from about 7 BPM to below 2 BPM when the sound level exceeds 49 dB. Sensing sound at this level is perceived acceptable, according to observations of the response of children participants and their parents' feedback, and users outside the car can barely hear the sound. Also, note that previous works use higher sound levels (reportedly 56 dB and 75 dB) than the SAS-based detection system. Nevertheless, a higher sound level is more favored in the SAS-based detection system as a relatively high sound level could benefit CPD applications since it promises a better chance to wake up a sleeping baby for more reliable detection via awake motion. As research reports that a sound level higher than 75 dB will disturb the infants, the default sound level is set as 50 dB. Based on real-world experiments with children, such a sound level appears to be tolerable to kids including infants and toddlers: It is received no cases to complain about the sound intrusiveness and annoyance.

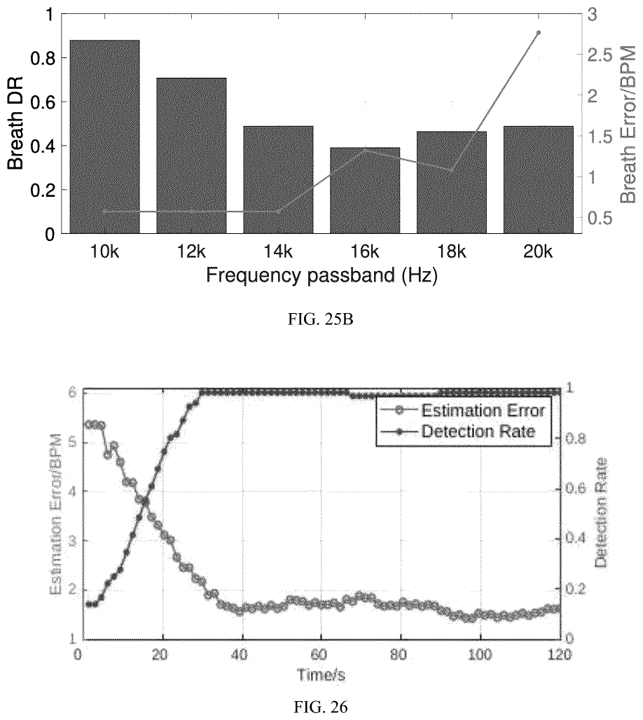

The band above 10 kHz is used by default in the experiments, which may still be intrusive to human cars. The performance with narrower and higher frequency bands is studied. To do so, the passband of the high-pass filter from 10 kHz to 22 kHz with a 2 kHz step is adapted. FIGS. 25A and 25B show that the SAS-based detection system retains a good performance until the passband exceeds 20 kHz. Larger bandwidths allow better performance, while the SAS-based detection system still performs well using 18-24 kHz, the commonly used inaudible band in the literature. Commodity devices like Google Nest start to support acoustic frequencies up to 30 kHz, which it is believed will become more common in the future. Such devices allow a sufficiently large and truly inaudible band across the age spectrum for non-intrusive acoustic sensing.

As sound speed depends on temperature, the SAS-based detection system was also tested under high temperatures. In the experiment, surrounding air is heated to about 120° F. and then let it naturally cool down in a warm room of about 70° F. The system is kept running during the process and show the breathing estimation results in FIG. 26. As seen, the SAS-based detection system failed to work when the devices overheat, but resumes excellent performance when they slightly cool down (after 30 seconds). A CPD system is expected to work before rather than after the car has heated up, as intervention actions are most effective right after the car is parked and locked. Therefore, it is believed that the SAS-based detection system's CPD effectiveness will not be affected even though its performance degrades under overly high temperatures.

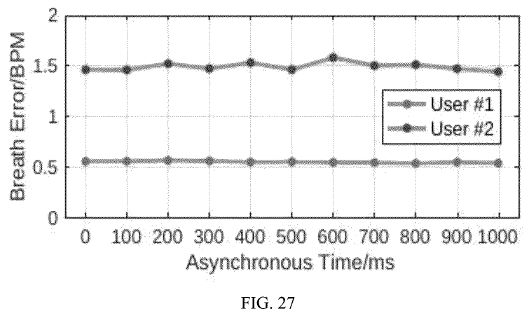

Large synchronization errors are introduced to show that the SAS-based detection system is resilient to phase offsets. Particularly, the starting point of the received signals is shifted by an amount of time ranging from 0 to 1 s with a step of 0.1 s. As shown in FIG. 27, the SAS-based detection system maintains similar accuracy without being affected by the time offsets, which confirms theoretical analysis.

Infants and toddlers usually have higher breathing rates than adults. The performance of the SAS-based detection system with respect to a range of breathing rates from 30 BPM to 60 BPM is evaluated. The breathing rate for each run is fixed by controlling the motor of the infant simulator. The results show insignificant differences for various breathing rates.

The system is benchmarked overhead on a desktop (Intel i7-11700 @ 4.9 GHZ CPU), a MacBook Air M1, and a Raspberry Pi 3 Model B+, on which the SAS-based detection system use 0.52 s, 0.73 s, and 3.97 s respectively to process 10 s of the data stream. The results show that the SAS-based detection system can run in real-time on embedded devices, promising its integration into existing car control systems.

A sufficient sampling rate of CSI is required to estimate speed. Given a sound frequency f with wavelength λ(f), a moving speed v is expected to experience a peak at the delay of

$$\\ τ = \frac{{x}_{0} λ ( f )}{2 π v} .$$

Assume that it needs at least Q samples to reliably detect a peak, which corresponds to a delay of τₘᵢₙ=Q/Fₛ. Then, it can derive the minimum sampling rate required to measure a speed of v by

$$\\ τ = \frac{{x}_{0} λ ( f )}{2 π v} > {τ}_{min} = Q / {F}_{s} ,$$

which implies

$$\\ {F}_{s} > \frac{2 π Qv}{{x}_{0} λ ( f )} .$$

In other words, the maximum speed can be calculated as

$$\\ v < \frac{{x}_{0} λ ( f ) {F}_{s}}{2 π Q} ,$$

which becomes about 0.1 m/s at f=20 kHz (wavelength 1.7 cm), about 0.2 m/s at f=10 kHz, and about 2 m/s at f=1 kHz, assuming Q=5 and a sampling rate of about 50 Hz (considering the sound speed of c=343 m/s). Using lower frequencies immediately allows to support higher speed, which however may suffer more from ambient noises. How to break down the sampling rate limitations and achieve estimation of daily speed (e.g., 0.5 m/s to 2 m/s) using pseudo-ultrasound frequencies remains worthwhile direction.

For breathing signals, since the periodicity is independent of subcarrier frequency, MRC can be directly performed across subcarriers. However, a further trick is needed to combine speed signals because, for acoustic signals from 10 kHz to 24 kHz, the difference in the wavelengths cannot be neglected (the wavelength at 10 kHz is approximately twice of that at 24 kHz). Recall

$$\\ \hat{v} = \frac{{x}_{0} λ ( f )}{2 π {τ}_{s}} .$$

Given the same speed v, the first local peaks of the ACF on different subcarriers will appear at different delays τₛ. Hence, to combine subcarriers for speed signals, it needs to first compensate the linear offsets due to different wavelengths. Specifically, the ACF\tilde{ρ}(f, τ) can be expressed as a unit linearly proportional to λ(f), i.e.,

$$\\ μ = \frac{τ}{λ ( f )} ,$$

and then average on \tilde{ρ}(f, μ). The operation is equivalent to scaling the ACF in the time lag dimension, which can be achieved by interpolation.

Here it shows a simple but stringent proof of that synchronization errors do not affect the SAS-based detection system. Denote the CIR measured under synchronization offsets as \tilde{h} (t):

\tilde{h}(t)=circshift(h(t),τ_{off})

where h (t) is the true CIR, τ_{off} is the timing offset caused by asynchronization, and circshift (.) represents circular shift. The time offsets correspond to phase shifts in the frequency domain. Thus, the asychronized CSI can be inferred as the following equation:

$$\\ \tilde{H} ( f ) = H ( f ) \cdot {e}^{{}_{} - j 2 π f {τ}_{off}}$$

where H(f) is the true CSI. Thus, |\tilde{H} (f)|=|H(f)| is obtained, meaning that the SAS-based detection system is resilient to synchronization errors.

The functional units and modules of the SAS-based detection system in accordance with the embodiments disclosed herein may be implemented using computing devices, computer processors, or electronic circuitries including but not limited to application specific integrated circuits (ASIC), field programmable gate arrays (FPGA), microcontrollers, and other programmable logic devices configured or programmed according to the teachings of the present disclosure. Computer instructions or software codes running in the computing devices, computer processors, or programmable logic devices can readily be prepared by practitioners skilled in the software or electronic art based on the teachings of the present disclosure.

All or portions of the methods in accordance to the embodiments may be executed in one or more computing devices including server computers, personal computers, laptop computers, mobile computing devices such as smartphones and tablet computers.

The embodiments may include computer storage media, transient and non-transient memory devices having computer instructions or software codes stored therein, which can be used to program or configure the computing devices, computer processors, or electronic circuitries to perform any of the processes of the present invention. The storage media, transient and non-transient memory devices can include, but are not limited to, floppy disks, optical discs, Blu-ray Disc, DVD, CD-ROMs, and magneto-optical disks, ROMs, RAMs, flash memory devices, or any type of media or devices suitable for storing instructions, codes, and/or data.

Each of the functional units and modules in accordance with various embodiments also may be implemented in distributed computing environments and/or Cloud computing environments, wherein the whole or portions of machine instructions are executed in distributed fashion by one or more processing devices interconnected by a communication network, such as an intranet, Wide Area Network (WAN), Local Area Network (LAN), the Internet, and other forms of data transmission medium.

The foregoing description of the present invention has been provided for the purposes of illustration and description. It is not intended to be exhaustive or to limit the invention to the precise forms disclosed. Many modifications and variations will be apparent to the practitioner skilled in the art.

The embodiments were chosen and described in order to best explain the principles of the invention and its practical application, thereby enabling others skilled in the art to understand the invention for various embodiments and with various modifications that are suited to the particular use contemplated.

What is claimed is:

1. A system for detecting presence of a subject in a vehicle cabin, comprising:
one or more transmitters, each configured to transmit an acoustic signal to the subject in the vehicle cabin; and
at least one receiver configured to receive a plurality of acoustic multipath signals scattered by the subject;
a controller configured to generate one or more driving signals to control the one or more transmitters to transmit the acoustic signal; and
a processor coupled with the controller and configured to receive the plurality of acoustic multipath signals from the receiver and process the plurality of acoustic multipath signals to detect presence of the subject in the vehicle cabin; and
wherein the presence of the subject is detected by:
extracting a plurality of channel impulse response (CIR) data from the plurality of received acoustic multipath signals;
aggregating the plurality of extracted CIR data to estimate acoustic channel state information (CSI);
obtaining an autocorrelation function (ACF) of the acoustic CSI based on a statistical acoustic sensing (SAS) model; and
performing one or more physiological activity monitoring on basis of the ACF to detect presence of the subject in the vehicle cabin.

2. The system of claim 1, wherein the acoustic CSI is given by:
H(f,t)=Σ_{i∈R}_{_{D}}Hᵢ(f,t)+Σ_{j∈R}_{_{S}}Hⱼ(f,t)+N(f,t);
where H(f, t) denotes the acoustic CSI, Hᵢ(f, t) denotes a component contributed by a ith scatterer, N(f, t) is a noise term with variance σ_{N}², and R_{S} and R_{D} denote a set of static and dynamic scatterers, respectively.

3. The system of claim 2, wherein the autocorrelation function (ACF) is given by:
$$\\ ρ ( f , τ ) = \frac{{\sum }_{i ∈ {R}_{D}} 2 π {σ}_{i}^{{}_{} 2} ( f ) + {σ}_{N}^{{}_{} 2} ( f ) δ ( τ )}{{\sum }_{i ∈ {R}_{D}} 2 π {σ}_{i}^{{}_{} 2} ( f ) + {σ}_{N}^{{}_{} 2} ( f )} {J}_{0} ( kv τ ) , for τ \neq 0$$
where ρ(f, τ) denotes the ACF of H(f, t) with time lag τ, δ(⋅) is the Dirac's delta function;
$$\\ {J}_{0} ( x ) = \frac{1}{2 π} {\int }_{0}^{2 π} exp ( - jx cos ( θ ) ) d θ$$
is the 0^{th}-order Bessel function of the first kind, v is the moving speed of the subject, and k is the wavenumber.

4. The system of claim 3, wherein the one or more physiological activity monitoring includes motion detection; and the motion detection is performed by:
calculating a channel gain of the acoustic CSI from the ACF;
comparing the channel gain against a threshold;
determining that motion is detected if the channel gain is equal or greater than the threshold.

5. The system of claim 3, wherein
the channel gain is associated with the ACF by:
g(f)=\tilde{ρ}(f,τ)=ρ(f,τ)+n(f,τ),
where g(f) denotes the channel gain; \tilde{ρ} (f, τ) is the sampled ACF calculated from a time series of CSI measurements with the noise term n(f, τ); and
the channel gain is approximated as:
$$\\ g ( f ) = \tilde{ρ} ( f , τ = 1 / {F}_{s} ) ,$$
where Fₛ is the CSI sampling rate.

6. The system of claim 1, wherein the one or more physiological activity detection includes breathing tracking; and the breathing tracking is performed by:
searching peaks in the autocorrelation function (ACF) over time corresponding to a cycle time of breathing; and
determining that breathing is detected and tracked if the peaks are found.

7. The system of claim 6, wherein
an optimized ACF is obtained by combining one or more autocorrelation function (ACF) corresponding to one or more subcarriers through a maximal ratio combining (MRC) algorithm; and
the one or more physiological activity detection includes breathing tracking and the breathing tracking is performed by:
searching peaks in the optimized ACF over time corresponding to a cycle time of breathing; and
determining that breathing is detected and tracked if the peaks are found.

8. The system of claim 1, wherein the one or more driving signals are modulated with a pseudo-noise sequence.

9. The system of claim 1, wherein the pseudo-noise sequence is a Kasami sequence.

10. The system of claim 1, further comprising a first high-pass filter applied on the transmitted acoustic signal and a second high-pass filter applied on the received acoustic signal.

11. A method for detecting presence of a subject in a vehicle cabin, the method comprising:
transmitting an acoustic signal to the subject in the vehicle cabin;
receiving a plurality of acoustic multipath signals scattered by the subject;
processing the plurality of acoustic multipath signals to detect presence of the subject in the vehicle cabin by:
extracting a plurality of channel impulse response (CIR) data from the plurality of received acoustic multipath signals;
aggregating the plurality of extracted CIR data to estimate acoustic channel state information (CSI);
obtaining an autocorrelation function (ACF) of the acoustic CSI based on a statistical acoustic sensing (SAS) model; and
performing one or more physiological activity monitoring on basis of the ACF to detect presence of the subject in the vehicle cabin.

12. The method of claim 1, wherein the acoustic CSI is given by:
H(f,t)=Σ_{i∈R}_{_{D}}Hᵢ(f,t)+Σ_{j∈R}_{_{S}}Hⱼ(f,t)+N(f,t);
where H(f, t) denotes the acoustic CSI, Hᵢ(f, t) denotes a component contributed by a ith scatterer, N(f, t) is a noise term with variance σ_{N}², and R_{S} and R_{D} denote a set of static and dynamic scatterers, respectively.

13. The method of claim 12, wherein the autocorrelation function (ACF) is given by:
$$\\ ρ ( f , τ ) = \frac{{\sum }_{i ∈ {R}_{D}} 2 π {σ}_{i}^{{}_{} 2} ( f ) + {σ}_{N}^{{}_{} 2} ( f ) δ ( τ )}{{\sum }_{i ∈ {R}_{D}} 2 π {σ}_{i}^{{}_{} 2} ( f ) + {σ}_{N}^{{}_{} 2} ( f )} {J}_{0} ( kv τ ) , for τ \neq 0$$
where ρ(f, τ) denotes the ACF of H(f, t) with time lag τ, δ(⋅) is the Dirac's delta function;
$$\\ {J}_{0} ( x ) = \frac{1}{2 π} {\int }_{0}^{2 π} exp ( - jx cos ( θ ) ) d θ$$
is the 0^{th}-order Bessel function of the first kind, v is the moving speed of the subject, and k is the wavenumber.

14. The method of claim 13, wherein the one or more physiological activity monitoring includes motion detection; and the motion detection is performed by:
calculate a channel gain of the acoustic CSI from the ACF;
comparing the channel gain against a threshold;
determining that motion is detected if the channel gain is equal or greater than the threshold.

15. The method of claim 13, wherein
the channel gain is associated with the ACF by:
g(f)=\tilde{ρ}(f,τ)=ρ(f,τ)+n(f,τ),
where g(f) denotes the channel gain; \tilde{ρ}(f, τ) is the sampled ACF calculated from a time series of CSI measurements with the noise term n(f, τ); and
the channel gain is approximated as:
$$\\ g ( f ) = \tilde{ρ} ( f , τ = 1 / {F}_{s} ) ,$$
where Fₛ is the CSI sampling rate.

16. The method of claim 11, wherein the one or more physiological activity detection includes breathing tracking; and the breathing tracking is performed by:
searching peaks in the autocorrelation function (ACF) over time corresponding to a cycle time of breathing; and
determining that breathing is detected and tracked if the peaks are found.

17. The method of claim 16, wherein an optimized ACF is obtained by combining one or more autocorrelation function (ACF) corresponding to one or more subcarriers through a maximal ratio combining (MRC) algorithm; and
the one or more physiological activity detection includes breathing tracking and the breathing tracking is performed by:
searching peaks in the optimized ACF over time corresponding to a cycle time of breathing; and
determining that breathing is detected and tracked if the peaks are found.

18. The method of claim 11, wherein the one or more driving signals are modulated with a pseudo-noise sequence.

19. The method of claim 11, wherein the pseudo-noise sequence is a Kasami sequence.

20. The method of claim 11, further comprising: applying filtering the transmitted acoustic signal and the received acoustic signal with a high-pass filter respectively.
